(19) United States
(12) Patent Application Publication
(10) Pub. No.: US 2024/0180583 A1
(43) Pub. Date: Jun. 6, 2024

(54) EXCISION CATHETER SYSTEM FOR AN AORTIC VALVE

(71) Applicant: Excision Medical, Inc., Malvern, PA (US)

(72) Inventors: David Alexander WOOD, Vancouver (CA); Janarthanan SATHANANTHAN, Vancouver (CA); Michael Austin DOTSEY, Malvern, PA (US); Francois Louis JOUIN, Malvern, PA (US); Jason Donald KEISER, Malvern, PA (US); Greg Alan WALTERS, Malvern, PA (US)

(73) Assignee: Excision Medical, Inc., Malvern, PA (US)

(21) Appl. No.: 18/285,764

(22) PCT Filed: Apr. 7, 2022
(86) PCT No.: PCT/US2022/023927

§ 371 Date: Oct. 5, 2023

Related U.S. Application Data
(60) Provisional application No. 63/176,507, filed on Apr. 19, 2021.

(51) Int. Cl. A61B 17/3205; A61B 17/00; A61B 17/32

(52) U.S. Cl. CPC A61B 17/3205; A61B 17/00234; A61B 2017/00292; A61B 2017/00353; A61B 2017/00367; A61B 2017/320064; A61B 2017/320069

(57) ABSTRACT

The present disclosure describes an excision catheter system including a cutting catheter and extraction catheter. The cutting catheter is configured to pierce and/or cut a leaflet and the extraction catheter is configured to hold and/or remove the excised leaflet portion.

### CROSS-REFERENCE TO RELATED APPLICATION

The present application claims the benefit of and priority to U.S. Provisional Application No. 63/176,507, filed Apr. 19, 2021, the entire disclosure of which is incorporated by reference into this application for all purposes.

### TECHNICAL FIELD

The present disclosure relates to an excision catheter system.

### BACKGROUND

Transcatheter aortic valve replacement (TAVR) is an alternative option for the treatment of patients with severe calcific aortic stenosis. Indeed, TAVR may become the preferred therapy for all patients irrespective of surgical risk. However, transcatheter heart valves (THV) may fail in the future and repeat intervention may be required. So-called redo-transcatheter aortic valve implantation (TAVI) or TAVR may lead to risks of coronary obstruction due to the leaflet of the failed valve being pushed up by the new valve and leading to obstruction of blood flow to the coronary arteries.

TAVR in failed surgical bioprostheses is common. However, TAVR in failed transcatheter bioprostheses (i.e., transcatheter heart valve-in-transcatheter heart valve) will also become increasingly common. In both situations there is a risk of coronary obstruction. The risk of coronary obstruction can be predicted with the use of cardiac computed tomography. If the predicted risk of coronary occlusion is high, then percutaneous valve-in-valve intervention may be prohibitive. In some cases, the cause of the coronary obstruction is related to the leaflets of the failed surgical or transcatheter heart valve that are pushed up and prevent flow of blood to the coronary arteries.

### SUMMARY

There is a need for systems, devices and procedures for leaflet laceration in failed transcatheter heart valves. An embodiment of the present disclosure includes an excision catheter system including a cutting catheter and extraction catheter. The cutting catheter is configured to pierce and/or cut a leaflet and the extraction catheter is configured to extract the leaflet and debris and bubbles, and in some instances steer and filter, while also holding and removing the excised leaflet portion.

Another embodiment of the present disclosure is an excision catheter system. The excision catheter system includes a cutting catheter with a leading end, a trailing end spaced from the leading end in a distal direction along a central axis, an inner channel that extends from the leading end toward the trailing end, and at least one cutting element. The cutting element has an insertion configuration, where the at least one cutting element is located inside the inner channel, and a cutting configuration, where a terminal end of the at least one cutting element is extendable out of the inner channel.

Another embodiment of the present disclosure is an excision catheter system. The excision catheter system includes an extraction catheter having a distal end, a proximal end, and a channel that extends from the distal end toward the proximal end. The excision catheter system includes an extraction member disposed in the channel of the extraction catheter. The extraction member has a retracted configuration, where the extraction member is located inside the channel of the extraction catheter, and an expanded configuration, where the extraction member is expanded and positioned outside the extraction catheter. The excision catheter system also includes a cutting catheter with a leading end, a trailing end spaced from the leading end in a distal direction, an inner channel that extends from the leading end toward the trailing end, and at least one cutting element. When the extraction member is in the expanded configuration the leading end is configured to pass through the extraction member outside of the extraction catheter.

Another embodiment is an excision catheter system that includes an extraction catheter having a distal end, a proximal end, and a channel that extends from the distal end toward the proximal end. The excision catheter system includes an extraction member disposed in the channel and configured to exit the channel and expand. The excision catheter system also includes a cutting catheter with at least one cutting element, the at least one cutting element being moveable relative to the extraction member and is responsive to electric energy.

Another embodiment of the present disclosure includes an excision catheter system, comprising with an excision catheter assembly. The excision catheter assembly has an extraction catheter having a distal end, a proximal end, and a channel that extends from the distal end toward the proximal end. The excision catheter assembly has an extraction member disposed in the channel. The extraction member has a retracted configuration, where the extraction member is located inside the channel of the extraction catheter, and an expanded configuration, where the extraction member is expanded and positioned outside the extraction catheter. The excision catheter assembly includes an introducer configured to slide within the channel of the extraction catheter. The introducer has a lumen sized and configured to receive a guidewire. The excision catheter system also includes a cutting catheter assembly having a cutting catheter with a leading end, a trailing end spaced from the leading end in a distal direction along a central axis, and an inner channel that extends from the leading end toward the trailing end. The cutting catheter assembly has at least one cutting element having an insertion configuration, where the at least one cutting element is located inside the inner channel, a deployed configuration, where a terminal end of the at least one cutting element is located distal to the leading end in the distal direction, and a cutting configuration where the terminal end of the at least one cutting element is offset in a direction that is perpendicular to the central axis.

Another embodiment of the present disclosure is a method that includes advancing a steerable catheter into an aortic arch toward an implanted valve. The method includes advancing at least one cutting element from within a cutting catheter to a location outside a leading end of the cutting catheter. The method also includes splaying the at least one cutting element into a splayed configuration at a leaflet of the implanted valve. The method also includes retracting the at least one cutting element when in the splayed configuration to lacerate a leaflet of the implanted valve, thereby forming a lacerated leaflet portion.

Another embodiment of the present disclosure is a method that includes advancing a steerable catheter into an aortic arch toward an implanted valve. The method includes deploying an extraction member from within a channel of the steerable catheter into an expanded configuration. The method also includes advancing at least one cutting element from within a cutting catheter to a location outside a leading end of the cutting catheter. The method includes lacerating a leaflet with the at least one cutting element to form a lacerated leaflet portion. The method also includes capturing the lacerated leaflet portion with the extraction member and collapsing the extraction member into a retracted configuration to capture the lacerated leaflet portion. The method includes retracting the extraction member into the steerable catheter with the extraction member retaining the lacerated leaflet portion.

Another embodiment of the present disclosure is a method that includes inserting a guidewire through a sheath positioned in an aorta. The method further includes advancing, along the guidewire and through the sheath, a steerable catheter, and an introducer into an aortic arch toward an implanted valve. The method also includes removing the introducer and the guidewire from the steerable catheter. The method includes optionally leaving the guidewire in place. The method further includes deploying an extraction member from within a channel of the steerable catheter into an expanded configuration. The method includes advancing a leading end of a cutting catheter through the steerable catheter and outside of the expanded extraction member. The method includes steering the leading end of the cutting catheter toward a base of a leaflet of the implanted valve. The method includes advancing a cutting element from an insertion configuration, within the cutting catheter, into a deployed configuration, outside of the cutting catheter, so that the cutting element extends distal to the leading end of the cutting catheter to pierce the leaflet. The method also includes splaying the cutting elements. The method includes causing the splayed cutting elements to cut the leaflet to form a lacerated leaflet portion. The method includes retracting the cutting element to pull the lacerated leaflet portion into the expanded extraction member and the steerable catheter. The method also includes collapsing the extraction member into a retracted configuration to capture the lacerated leaflet portion. The method includes retracting the extraction member into the steerable catheter in the proximal direction with the extraction member retaining the lacerated leaflet portion.

### BRIEF DESCRIPTION OF THE DRAWINGS

The foregoing summary, as well as the following detailed description of illustrative embodiments of the present application, will be better understood when read in conjunction with the appended drawings. For purposes of illustrating the present application, the drawings show exemplary embodiments of the present disclosure. It should be understood, however, that the present disclosure is not limited to the precise arrangements and instrumentalities shown in the drawings. In the drawings:

FIG. 1 depicts a schematic version of the aortic arch including an example TAVR valve implanted at the aortic annulus;

FIG. 2A is a schematic plan view of an excision catheter assembly according to an embodiment of the present disclosure and a TAVR sheath in the descending aorta;

FIG. 2B is a cross-sectional view of an excision catheter of the excision catheter assembly shown in FIG. 2A;

FIG. 3 is a schematic plan view of the excision catheter assembly shown in FIG. 2A, with the introducer removed and the extraction member expanded;

FIG. 4 is a schematic plan view of a cutting catheter assembly according to an embodiment of the present disclosure;

FIG. 5 is a schematic plan view of the cutting catheter assembly shown in FIG. 4, showing the cutting element in a splayed configuration;

FIG. 6 is a perspective schematic view of a cutting element according to an embodiment of the present disclosure;

FIG. 7 is another perspective schematic view of the cutting element shown in FIG. 6;

FIG. 8 is a perspective schematic view of the cutting element shown in FIG. 6, illustrating the cutting element in a splayed configuration;

FIG. 9 is a perspective schematic view of the cutting element shown in FIG. 7, illustrating the cutting element in a splayed configuration;

FIG. 10 is a schematic plan view of a distal portion of an introducer according to an embodiment of the present disclosure;

FIG. 11 is a schematic plan view of a distal portion of an introducer according to another embodiment of the present disclosure;

FIG. 12 depicts the placement of a guidewire tip to the base of the leaflet aligned with the right coronary ostia, as placed through a TAVR sheath located in the descending aorta (not shown);

FIG. 13 depicts the advancement of the steering extraction catheter and introducer, in combination, over the guidewire to a location in the ascending aorta;

FIG. 14 illustrates the steering extraction catheter after removal of the introducer and guidewire from the catheter;

FIG. 15 depicts the deployment of the excision filter basket, which abuts the walls of the aorta or existing TAVR valve and acts to filter blood ejected from the heart;

FIG. 16 depicts the advancement of the cutting catheter from within the steering extraction catheter to the base of the leaflet;

FIG. 17 depicts the advancement of the cutting tip with dual cutting elements from within the cutting catheter;

FIG. 18 depicts the cutting of the leaflet during retraction of the cutting catheter from within the hole pierced as shown in FIG. 16;

FIG. 19 illustrates the withdrawal of the excision filter basket following removal of the cutting catheter, illustrating the capture of the leaflet portion liberated in FIG. 18;

FIG. 20 illustrates a view of the damaged TAVR valve leaflets from the perspective of the ascending aorta down in the heart, showing the position of the wire tip at the base of the leaflet;

FIG. 21 shows the hole created by the activation of the pierced tip of the passing steering extraction catheter;

FIG. 22 shows the cutting wye with the cutting tips hooked on the damaged leaflet, at the location of the pierced hole;

FIG. 23 depicts how the leaflet would be cut by the splaying of the cutting tips on retraction;

FIG. 24 depicts the fully cut leaflet with the leaflet portion missing; and

FIG. 25 illustrates retraction of the cutting catheter to collapse the cutting wye tip.

### DETAILED DESCRIPTION OF ILLUSTRATIVE EMBODIMENTS

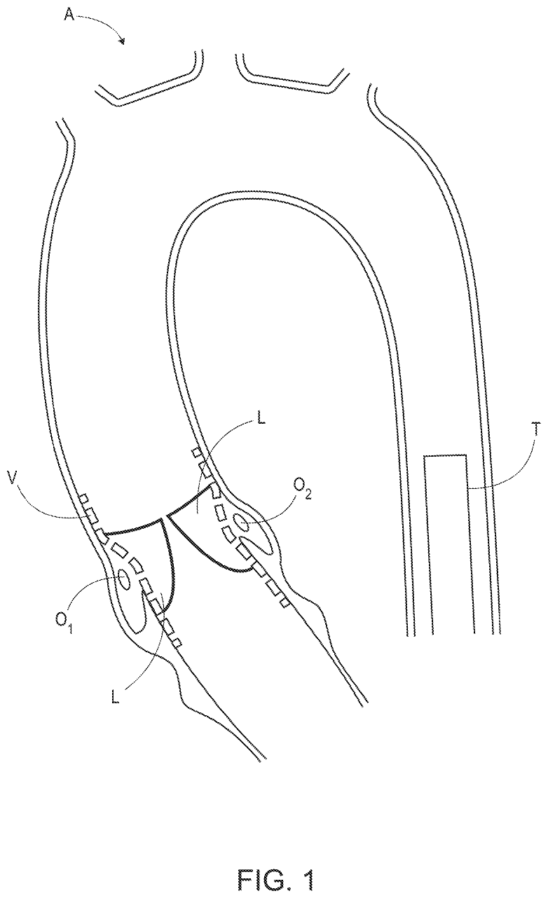

FIGS. 1-5 illustrates an excision catheter system 10 used to facilitate the cutting and removal of leaflet portions L from an implanted valve V in an aorta A. FIG. 1 specifically illustrates an aorta A and its aortic arch. Systems and methods as set forth in the present disclosure may be used to access, cut, and remove portion of leaflets or other tissue or deposits from an implant valve in an aorta. As shown, the aortic arch includes exemplary TAVR valve V implanted at the aortic annulus having damaged leaflets L that fail to coapt, and further details the location of the coronary ostia O₁, O₂, coronary vessels, and the arteries above the ascending aorta. A TAVR sheath T, used to access the valve V, is shown in the descending aorta. While a TAVR is shown, embodiments of the present disclosure may be used with surgical valves as well.

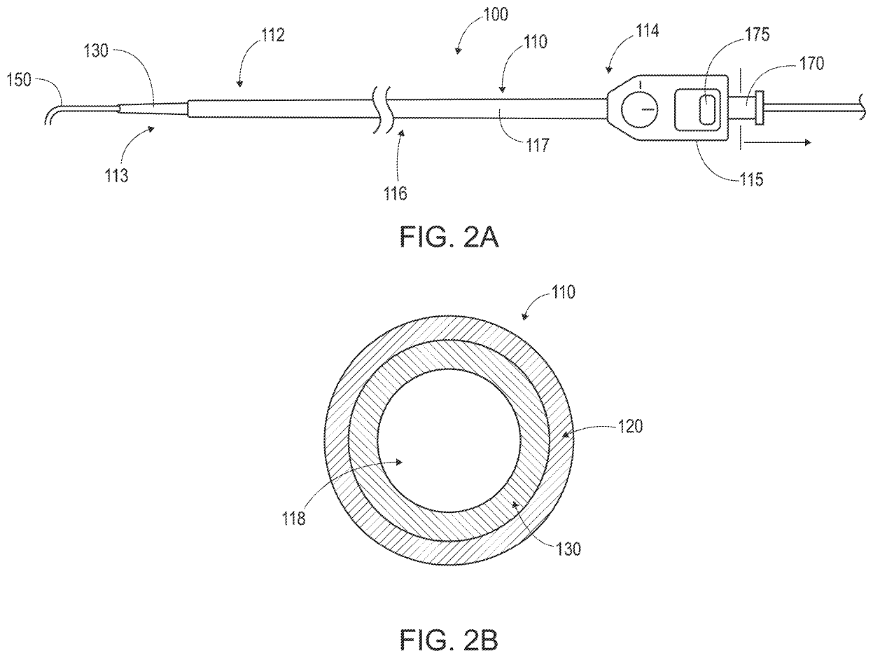

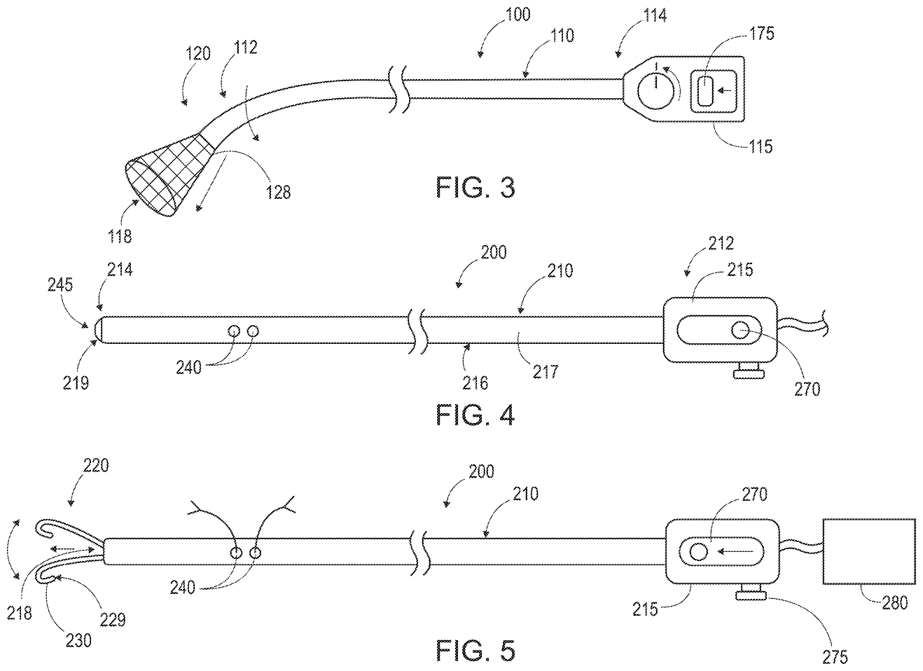

The systems, devices, and method as described herein are configured to provide access to, and the ability to safely remove, portion of the implant valve structure. As shown in FIGS. 2-5, an excision catheter system 10 may include an excision catheter assembly 100 (FIGS. 2A, 2B and 3) and a cutting catheter assembly 200 (FIGS. 4 and 5). The systems as described and shown in the figures are used for excising a portion of the leaflet of a valve. The excision catheter assembly 100 may be referred to as an outer catheter assembly while the cutting catheter assembly 200 may be referred to as an inner catheter assembly that is slidable within the outer catheter assembly. Both the outer catheter assembly 100 and the inner catheter assembly 200 can slide within a TAVR sheath. Accordingly, an extraction catheter 110 of the excision catheter assembly 100 may be referred to as an outer catheter while a cutting catheter 210 of the cutting catheter assembly 200 may be referred to as an inner catheter that is slidable within the outer catheter.

The excision catheter assembly 100 (FIGS. 2A, 2B and 3) includes an extraction catheter 110, an extraction member 120, and an introducer 130. The extraction catheter 110, extraction member 120, and the introducer 130, as shown in FIGS. 2A, 2B and 3, are inserted through the TAVR sheath as an assembled unit. The excision catheter assembly 100 is configured to present the extraction member 120, sometime referred to as an excision basket and filter, into the ascending aorta and cause the extraction member 120 to expand as explained further below. The excision catheter assembly 100 can be steered or guided into a position proximate the leaflet of the valve. In this regard, the extraction catheter 110 may be referred to also as a steering catheter. In such an example, its distal tip 113 can be steered or guided into position as needed to present the extraction member 120 and cutting catheter assembly 200 proximate the valve. Thus, the excision catheter assembly 100 also provides a means for the cutting catheter assembly 200 to access the implanted valve.

The cutting catheter assembly 200, as shown in FIGS. 4 and 5, includes a cutting catheter 210 and at least one cutting element 220. The cutting catheter assembly 200 may be inserted through the extraction catheter 110 once the introducer 130 is removed, as further described below. The cutting catheter 210 may be designed to cut the leaflet with cutting tip, cutting edge or blade, or via electrical energy. In an embodiment where electrical energy is used to lacerate the leaflet, the system may include an electrosurgical unit 280 (FIG. 5) configured to supply the electric energy to the at least one cutting element 220.

Cutting or severing the tissue with electrical energy, e.g., via an RF unit (e.g., electrosurgical unit 280), causes the tissues, blood, and water, etc., to vaporize. This, in turn, may cause formation of bubbles and other debris that may need to be extracted or removed from the aorta. More specifically, what is generated in the aorta responsive to RF laceration may likely be a combination of water vapor, char, smoke, oxygen, nitrogen, carbon dioxide, etc. Vaporizing tissue and arcing through blood may liberate all of these components, which could indicate a need to manage the capture and removal, i.e., extraction of these components.

The cutting catheter 210 further comprises a retention member 230 configured to retain an excised leaflet. In such an embodiment, the retention member 230 can be a harpoon, grasper, hooks, vacuum, etc., that is designed to snag and hold the leaflet portion.

The illustrated assemblies are shown as separate components, one for cutting a leaflet and the second removing the cut leaflet from the aorta. However, it is possible that the two illustrated assemblies may be combined into a single excision catheter system 10 that can both cut and remove the cut tissue. In one example, the excision catheter assembly 100 and the cutting catheter assembly 200 are configured to be combined and inserted into the implanted TAVR sheath as a single unit. In such an example, the handle of the system 10 is configured to facilitate the control of the various components and subcomponents, via actuators and the like.

The excision catheter assembly 100 includes an extraction catheter 110, an extraction member 120, and an introducer 130. The extraction catheter 110 may include a hub 115 at its proximal end 114 and an elongated body 116 coupled to the hub. The elongated body 116 includes a shaft portion 117, a secondary curve, a primary curve, one or more radiopaque markers, and a distal tip 113. The primary and secondary curves are not illustrated in the drawings. The distal tip 113 defines the distal end 112 of the extraction catheter 110. The extraction catheter 110 includes a channel 118 that extends from the proximal end to the distal end of the elongated body 116. The channel 118 is sized to contain or receive therethrough all or portions of the introducer 130 and/or cutting catheter assembly 200. The channel 118 is also sized to receive other surgical devices therethrough. For example, the extraction catheter 110 can receive a guidewire 150 such that an over-the-wire technique may be used. That is, a guidewire 150 can be placed through the valve structure into the left ventricle and the extraction catheter 110 inserted over the guidewire into position. In an alternative embodiment, the extraction catheter 110 may include one or more skive ports (not shown) that can be used to receive the guidewire therethrough. Such skive ports may be disposed toward or along an outer surface of the extraction catheter 110. In yet another embodiment, the guidewire 150 may not extend through the valve structure into the ventricle. The extraction catheter 110 may still slide over or along the guidewire 150, but without the benefit of having the guidewire cross through the valve structure.

In cross-section, extraction catheter 110 may include an inner liner, a middle reinforcing layer (e.g., a braid), and an outer layer or outer jacket. In another embodiment, the extraction catheter 110 would also be able to accommodate different shaped inner catheters to achieve a suitable relationship of the distal tip 113 to the leaflet. For example, this configuration may provide for functionality similar to the use of a 5 F/6 F 120 mm IM catheter inside an AL type catheter, i.e., a mother and daughter technique. The extraction catheter 110 may be configured to transition in response to operator input to assume different degrees of flexion of the distal tip 113 to account for different patient anatomy.

The longitudinal shape of the catheter can vary as needed. For instance, the extraction catheter 110 can have a shape according to the Amplatz Guide that includes, but is not limited to AL-1, AL-2, AL-3, AL-4, etc. Other common shapes are possible as well. In one example, the catheter may have an outer cross-sectional dimension sized for insertion into the aorta. For instance, the catheter may be either 12 French or 14 French. However, larger or smaller sized catheters may be used in certain instances. The catheter tip or distal tip 113 may be deflectable or bendable as needed to steer the distal tip into position. The extraction catheter 110 may also be configured to accommodate different shaped inner catheters.

The extraction member 120 may be any device that is expandable and can receive or retain lacerated tissue therein. In one example, the extraction member 120 may be a basket, filter, or other device 128 that permits blood flow therethrough but can retain lacerated tissue as needed. In one example, the extraction member 120 is also configured to permit blood flow therethrough while also capturing bubbles released/generated by cutting the leaflet with RF energy as described herein. This so-called filter 128 may be a material with pore sizes that are typically between 80 microns and 120 microns. In one example, the pore size is about 100 microns. It should be appreciated that pore size as used herein is an average pore size and the pores in the material forming the material may have a size less than 100 microns or more than 100 microns. In other words, the filter 128 will have a pore size distribution whereby a substantial majority of the pores are no larger than about 100 microns. While the extraction member 120 may function as a filter, the extraction member can also function as a temporary valve to limit blood pressure or blood flow as needed.

In accordance with an embodiment, the extraction catheter 110 may have one or more actuators. In one example, the extraction catheter 110 has a first actuator 170 configured to steer the proximal end 114 of the extraction catheter into a desired position (FIG. 2A), and a second actuator 175 configured to cause the extraction member 120 to transition between the insertion configuration (FIG. 2A) and the expanded configuration (FIG. 3). It is to be understood that one actuator may be used as needed.

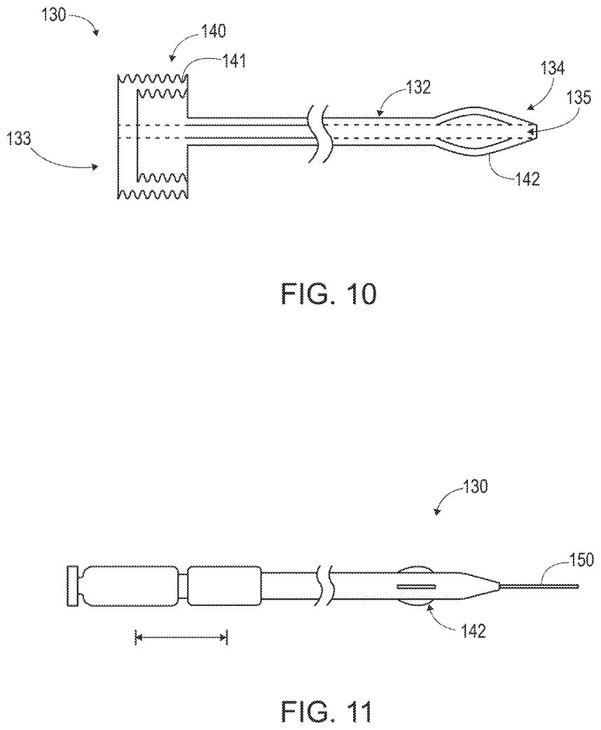

The introducer 130 has an elongated body 132, a proximal end 133, a distal end 134, and channel 135 that extends from the proximal end 133 to the distal end 134. The introducer 130 has a lumen sized and configured to receive a guidewire 150. The introducer 130 is removable from the channel 118 of the extraction catheter 110 such that the cutting catheter 210 is insertable into the channel 118 of the extraction catheter 110. It should be appreciated that the introducer 130 and extraction catheter 110 are configured to curve along with an arch of an ascending aorta when assembled together. In one embodiment, the introducer 130 has an expandable member 140 configured to maintain slidable engagement inside the extraction catheter 110 (FIGS. 10 and 11). In one example, the expandable member 140 is threadably engaged with the extraction catheter 110 via a plurality of threads 141, as shown in FIG. 10. In another example, the expandable member 140 is a plurality of flexible wires 142 configured to expand toward the inner surface of the extraction catheter 110, as shown in FIGS. 10 and 11.

The entire assembly may be sized to fit within a TAVR sheath. For example, the excision catheter assembly 100 may have an outer diameter, measured perpendicular to a central axis thereof, up to about 14 F. The inner diameter of the introducer 130 is sized to fit around a guidewire 150 and may be at least 0.035 inches. It could vary among this size as needed.

The cutting catheter assembly 200 includes a cutting catheter 210 and at least one cutting element 220. The cutting catheter 210 may include a trailing end 212, a leading end 214, and inner channel 218 that extends therethrough. The cutting catheter 210 may include a hub 215 at its trailing end 212 and an elongated body 216 coupled to the hub 215 that defines the leading and trailing ends. The elongated body 216 includes a shaft portion 217, a secondary curve, a primary curve, one or more radiopaque markers, and a distal tip 219. The primary and secondary curves are not illustrated in the drawings. The distal tip 219 defines the leading end 214 of the cutting catheter 210, and may define a piercing tip 245, as explained below. The cutting catheter 210 includes inner channel 218 that extends from the leading end 214 toward the trailing end 212 of the elongated body 216. The inner channel 218 is sized to contain or receive therethrough other surgical devices therethrough. For example, the cutting catheter 210 can receive a guidewire such that an over-the-wire technique may be used.

The cutting catheter 210 has at least one port 240 that extends to the inner channel 218. As shown, the at least one port 240 could be two or more as needed. The port or ports 240 are spaced a distance from the leading end 214 that is less than a distance between the at least one port 240 and the trailing end 212. In other words, they are positioned toward the leading end 214 of the cutting catheter 210. These ports 240 are intended to a) allow removal of the air and other debris after cutting, and throughout, to provide for hemodynamic monitoring of the blood pressure in the ascending aorta. For instance, when the leaflets get cut, the destruction of the aortic valve will quickly lead to decompensation of coronary output, which is monitored by a local lumen. The catheter system, may, in turn, include a luer fitting on the handle for monitoring and bubble removal. Bubble and debris removal can happen via an active ‘vac lok’ syringe (pull a vacuum with a syringe and the handle locks in place so holding the user is not required) on the port evacuating 50-100 ml of blood/air, and/or upon removal of the cutting catheter assembly 200 as it pistons the blood out of the extraction catheter 110 on removal. In accordance with an embodiment, one or more of the extraction catheter 110 and the cutting catheter 210 may include a hemostasis valve to minimize fluid loss and backflow during a procedure.

In cross-section, cutting catheter 210 may include an inner liner, a middle reinforcing layer (e.g., a braid), and an outer layer or outer jacket. In another embodiment, the cutting catheter 210 would also be able to accommodate different shaped inner catheters to achieve a suitable relationship of the distal tip 113 to the leaflet. For example, this configuration may provide for functionality similar to the use of a 5 F/6 F 120 mm IM catheter inside an AL type catheter, i.e., a mother and daughter technique. The cutting catheter 210 may be configured to transition in response to operator input to assume different degrees of flexion of the distal tip 113 to account for different patient anatomy.

The longitudinal shape of the catheter can vary as needed. For instance, the cutting catheter can have a shape according to the Amplatz Guide that includes, but is not limited to AL-1, AL-2, AL-3, AL-4, etc. Other common shapes are possible as well. In one example, the catheter may have an outer cross-sectional dimension sized for insertion into the aorta. For instance, the catheter may be up to about 10 French or 11 French. However, larger or smaller sized catheters may be used in certain instances. The catheter tip may be deflectable or bendable as needed. The cutting catheter 210 may also be configured to accommodate different shaped inner catheters.

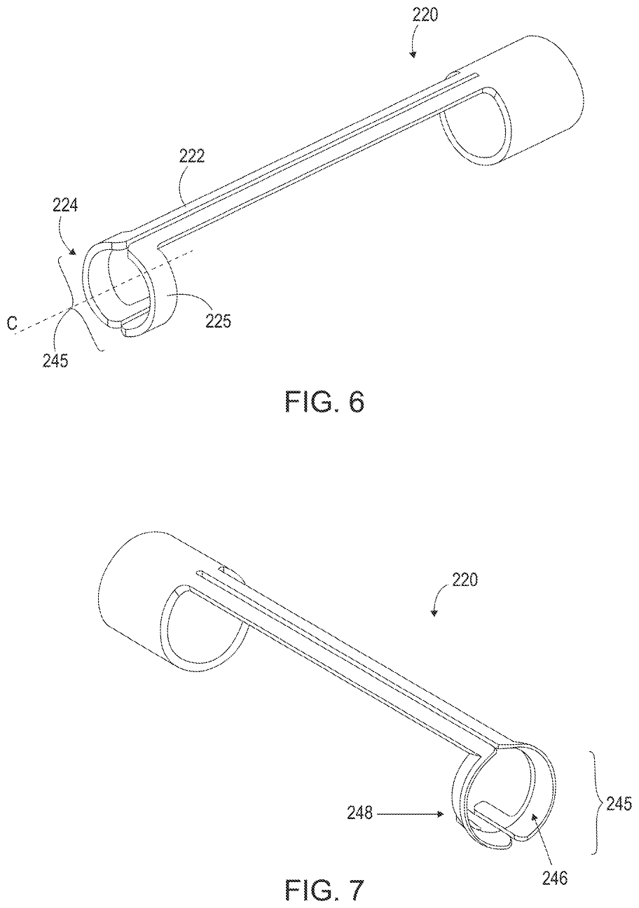

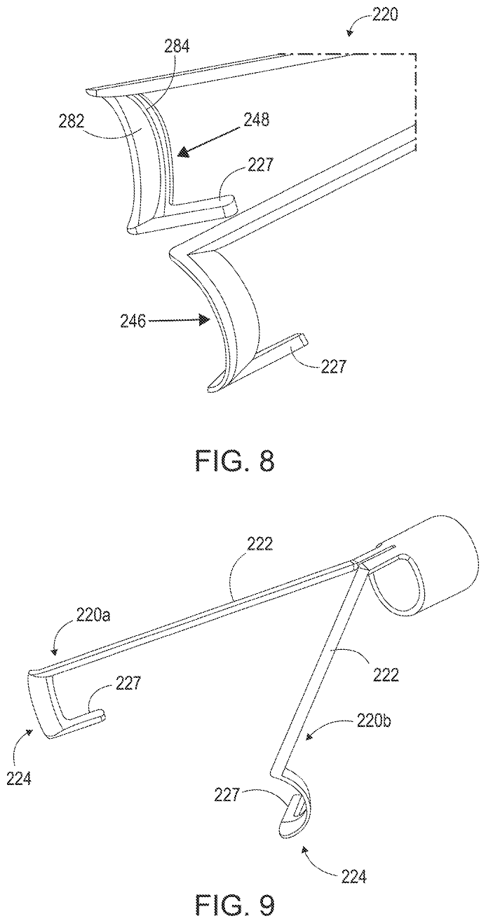

FIGS. 6-9 illustrate one embodiment of a cutting element 220. In one example the cutting element 220 may be pair of cutting elements 220A-B that are configured to separate or splay apart when needed to facilitate laceration of the leaflet. For purposes of clarity, the cutting elements will interchangeably be identified as 220 or 220A-B throughout the present disclosure. Each cutting element 220 includes an elongate shaft 222 and a cutting hook 224 that define terminal ends 229 of the cutting element 220 and extends from the elongate shaft. In accordance with an embodiment, the cutting hook 224 includes a leg 227 that extends substantially perpendicularly to the elongate shaft 222 (FIG. 9). The cutting hook 224 has a curved body 225 that curves around a central axis C of the cutting catheter 210. The curved body 225 defines a forward cutting edge 246 and a rearward cutting edge 248. When the pair of cutting elements 220A-B are in the insertion configuration, the pair of cutting hooks 224 are aligned to define a piercing tip 245 at a distal-most end of the cutting catheter 210, as shown in FIGS. 6 and 7. Furthermore, the distal-most terminal ends 229 of the pair of cutting elements 220A-B are substantially splayed apart when in the cutting configuration, as shown in FIGS. 8 and 9. More specifically, the terminal ends 229 of the pair of cutting elements 220A-B are configured to splay apart along a direction that is angularly offset to a central axis C of the cutting catheter 210. In accordance with an embodiment, the terminal ends 229 of the pair of cutting elements 220A-B are configured to splay apart along a direction that is substantially perpendicular to the central axis C of the cutting catheter 210. It should be appreciated that the cutting element 220 can be designed to pierce the leaflet with the terminal ends 229 adjacent to each other and then cut the leaflet when the terminal ends are splayed apart. For instance, the leading end 214 of the cutting catheter 210 can define a piercing tip 245 that extends around its perimeter (and about the central axis) while separate cutting elements 220A-B are used to extend out of the leading end 214, splay, then sever or cut the leaflet when splayed. Alternatively, the cutting elements 220A-B can define the piercing tip 245 when in the insertion configuration, as discussed above. In accordance with an embodiment, the cutting element 220 can include a movable shield (not shown) configured to selectively expose a portion of the cutting element to a leaflet to be cut.

As shown in FIGS. 5-7, the cutting catheter 210 may have one or more actuators. In one example, the cutting catheter 210 has a first actuator 270 configured to cause the at least one cutting element 220 to transition from the insertion configuration (FIGS. 6 and 7) into the deployed configuration (FIG. 5). In this example, the cutting element can automatically splay apart upon exit of the catheter. In another embodiment, a second actuator 275 is configured to cause the at least one cutting element 220 to transition between the deployed configuration (FIG. 5) and the cutting configuration (FIGS. 8 and 9). More specifically, the second actuator can maintain the cutting elements in a generally parallel state until the correct position is attained then permit the cutting elements to splay apart.

In an example, the cutting elements are designed to sever the tissue using cutting edges, as described above.

In another embodiment, the cutting elements 220 are electrodes that are responsive to electrical energy and supply electrical energy to the tissue to facilitate laceration, i.e., the cutting elements are configured for electrosurgical cutting. In such an example, an electrosurgical unit 280 may be used to supply electrical energy to the cutting element 220. Without being bound by any particular theory, the electrosurgical cutting of tissue depends on creating the correct current density at the location of the desired cutting location within the tissue. Thus, controlling the exposed surface area of the cutting elements 220 is a consideration, with the current emanating from just the piercing tip 245 during the pierce, and then just at the cutting hooks 224 on retraction during the cutting of the leaflet. This can be accomplished by several methods including 1) a complete coating of metal electrodes with the coating removed at the desired location, 2) electrodes embedded such that only the desired surface area is exposed, or 3) covering and uncovering (shielding and unshielding) portions of the electrode as desired to control the exposed surface area and hence allowing the ability to better control current density at the desired location.

For example, as shown in FIG. 8, the cutting element 220 includes a coated portion 282 and an exposed portion 284, such that, the coated portion and the exposed portion have different conductivities from each other. In accordance with an embodiment, the coated portion 282 and exposed portion 284 are positioned at the forward cutting edge 246 and the rearward cutting edge 248 of the cutting element 220. Although the coated portion 282 and exposed portion 284 are described as having different conductivities, it is to be understood that each coated portion can be non-conductive, and each exposed portion can be conductive.

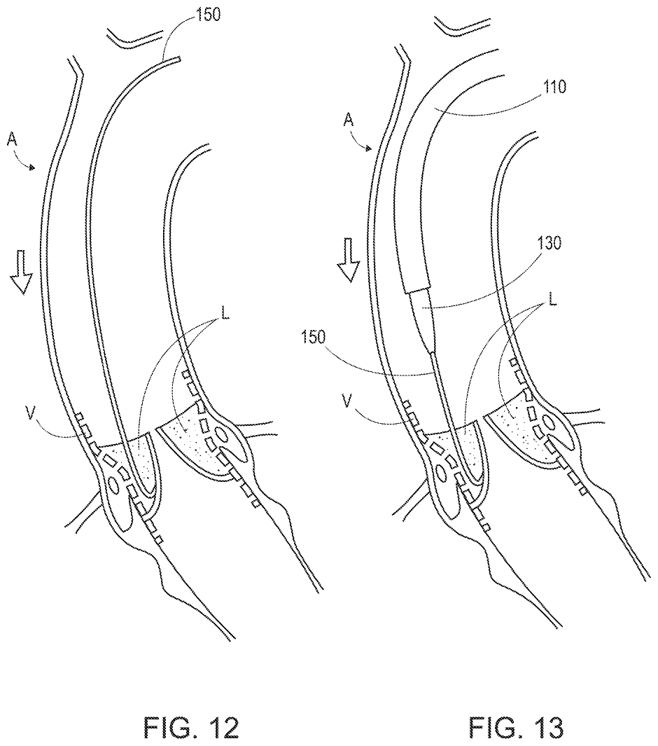

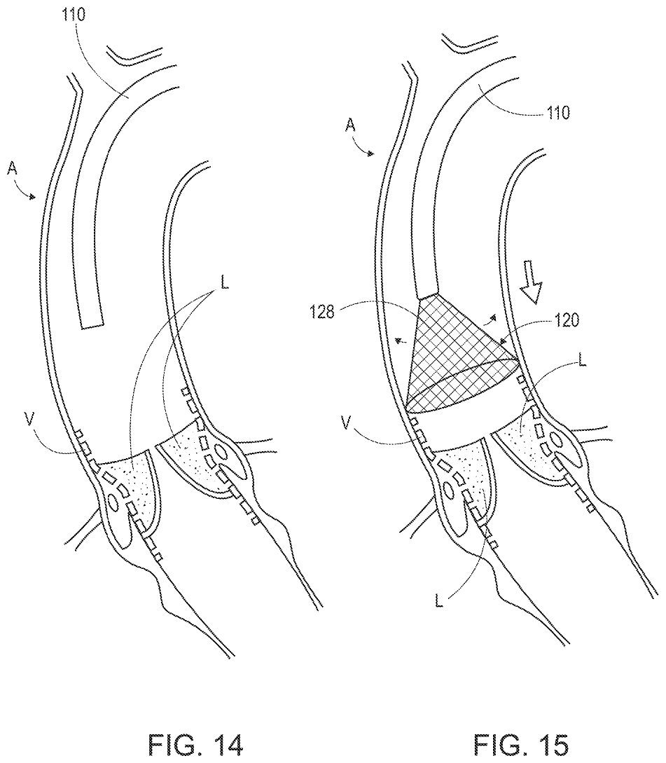

FIGS. 12-25 illustrate different embodiments of the method for cutting and removing a leaflet portion. FIG. 12 depicts the placement of a guidewire tip to the base of the leaflet aligned with the right coronary ostia, as placed through a typical TAVR sheath located in the descending aorta. FIG. 13 illustrates the advancement of the steering catheter 110 (i.e., extraction catheter) and introducer 130 over the guidewire 150 to a location in the ascending aorta. FIG. 14 illustrates the steering catheter 110 after removal of the introducer 130 and guidewire 150 from the steering catheter 110. FIG. 15 depicts the deployment of the excision filter basket 128, which abuts the walls of the aorta or existing TAVR valve and acts to filter blood ejected from the heart.

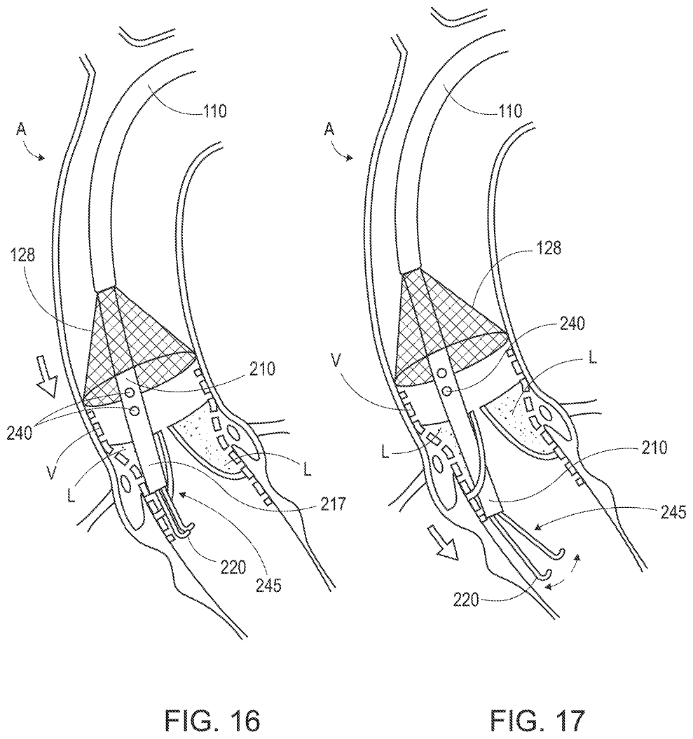

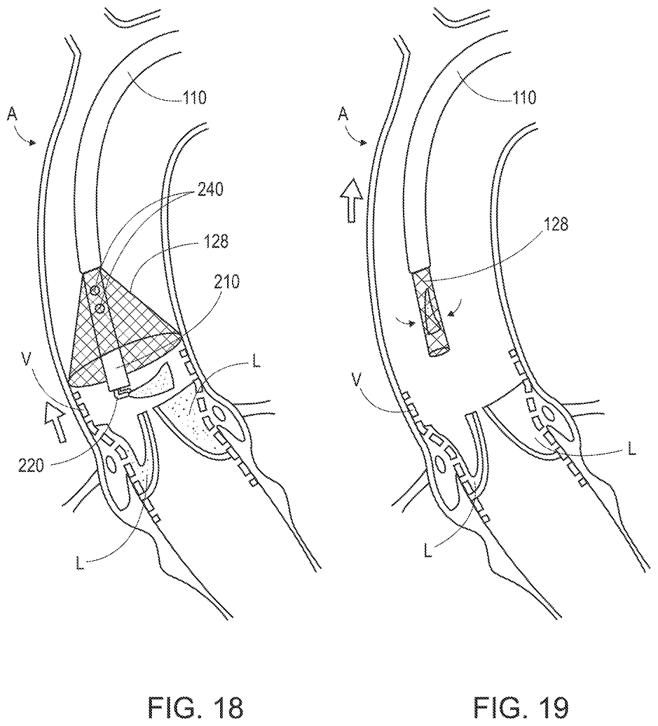

FIG. 16 depicts the advancement of the cutting catheter 210 from within the steering catheter 110 to the base of the leaflet, which is then energized at the distal tip 113 as shown in FIG. 2A to pierce and pass through the base of the leaflet. Advancement of the cutting catheter 210 exposes extraction ports 240 in the shaft portion 217 of the cutting catheter. FIG. 17 depicts the advancement of the cutting wye tip 245 (i.e., piercing tip) with dual cutting elements 220 from within the cutting catheter 210. Shown is the tip 245 nearly fully splayed as would occur during drawback of the cutting tip 245. FIG. 18 depicts the cutting of the leaflet during retraction of the cutting catheter 210 from within the hole pierced as shown in FIG. 16, resulting in a portion of leaflet removed from the existing damaged TAVR valve. Extraction of debris from the steering catheter 110 through the extraction ports 240 may occur in this position. FIG. 19 illustrates the withdrawal of the excision filter basket 128 following removal of the cutting catheter 210, illustrating the capture of the leaflet portion liberated in FIG. 18.

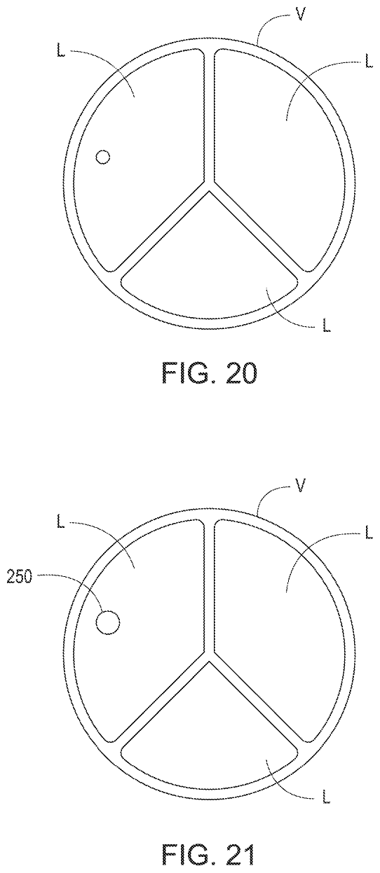

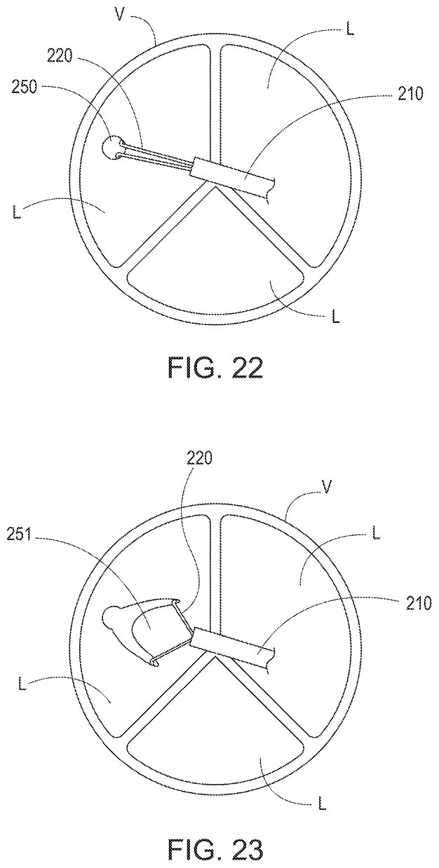

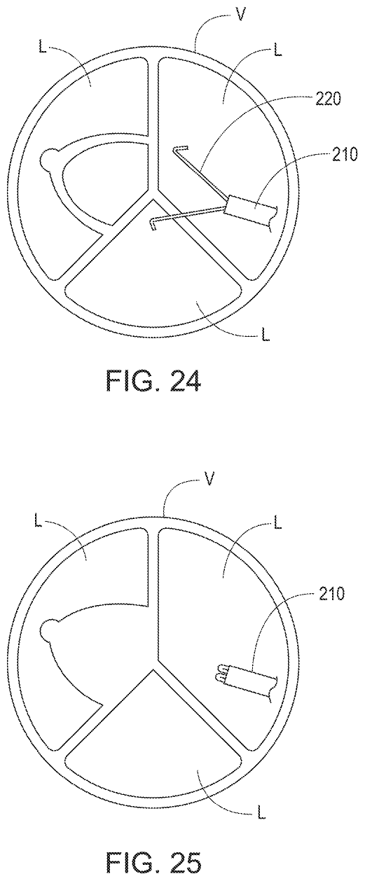

The present disclosure includes various embodiments of extracting a portion of a leaflet of a valve, e.g., a surgical valve or TAVR. The method may include inserting a guidewire 150 through a sheath positioned in an aorta. Then, a user can advance, along the guidewire 150 and through the sheath, a steering catheter 110, and an introducer 130 into an aortic arch toward an implanted valve. Next, the method includes removing an introducer 130 and guidewire 150 from the steering catheter 110. It should be appreciated that in some instances, the guidewire 150 may remain in place at this phase of the procedure to extend across or through the valve structure into the ventricle. The method also includes deploying an extraction member 120 from within a channel 118 of steering catheter 110 into an expanded configuration. Then, a user can advance a leading end 214 of a cutting catheter 210 through the steering catheter 110 and outside of the expanded extraction member 120. The method may include steering the leading end 214 of the cutting catheter 210 toward a base of a leaflet of the implanted valve. A user may then advance a cutting element 220 from an insertion configuration, within the cutting catheter 210, into a deployed configuration, outside of the cutting catheter, so that the cutting element extends distal to the leading end 214 of the cutting catheter to pierce the leaflet (FIGS. 20 and 21) and create a hole 250. The cutting elements are then splayed. As shown in FIG. 22, the cutting elements 220 are hooked onto the damaged portion (i.e., hole 250) of the leaflet. The method may then include causing the splayed cutting elements 220 to cut the leaflet to form a lacerated leaflet portion 251 (FIG. 23). The user may then retract the cutting element 220 to pull the lacerated leaflet portion into the expanded extraction member 120 and the steering catheter 110. As a result, the leaflet remains with the lacerated leaflet portion removed (FIG. 24). The method may include collapsing the extraction member 120 into a retracted configuration to capture the lacerated leaflet portion within the extraction member (FIG. 25). The method may include retracting the extraction member 120 into the steering catheter 110 in the proximal direction with the extraction member retaining the lacerated leaflet portion.

It will be appreciated by those skilled in the art that various modifications and alterations of the present disclosure can be made without departing from the broad scope of the appended claims. Some of these have been discussed above and others will be apparent to those skilled in the art. The scope of the present disclosure is limited only by the claims.

1. (canceled)

2. An excision catheter system, comprising:
an extraction catheter having a distal end, a proximal end, and a channel that extends from the distal end toward the proximal end;
an extraction member disposed in the channel of the extraction catheter, the extraction member having a retracted configuration, where the extraction member is located inside the channel of the extraction catheter, and an expanded configuration, where the extraction member is expanded and positioned outside the extraction catheter; and
a cutting catheter with a leading end, a trailing end spaced from the leading end in a distal direction, an inner channel that extends from the leading end toward the trailing end, and at least one cutting element, wherein when the extraction member is in the expanded configuration the leading end is configured to pass through the extraction member outside of the extraction catheter.

3. An excision catheter system, comprising:
an extraction catheter having a distal end, a proximal end, and a channel that extends from the distal end toward the proximal end;
an extraction member disposed in the channel and configured to exit the channel and expand; and
a cutting catheter with at least one cutting element, the at least one cutting element being moveable relative to the extraction member and is responsive to electric energy.

4. An excision catheter system, comprising:
a) an excision catheter assembly having:
an extraction catheter having a distal end, a proximal end, and a channel that extends from the distal end toward the proximal end;
an extraction member disposed in the channel, the extraction member having a retracted configuration, where the extraction member is located inside the channel of the extraction catheter, and an expanded configuration, where the extraction member is expanded and positioned outside the extraction catheter; and
an introducer configured to slide within the channel of the extraction catheter, the introducer having a lumen sized and configured to receive a guidewire; and
b) a cutting catheter assembly having:
a cutting catheter with a leading end, a trailing end spaced from the leading end in a distal direction along a central axis, and an inner channel that extends from the leading end toward the trailing end; and
at least one cutting element having a terminal end, the at least one cutting element having an insertion configuration, where the at least one cutting element is located inside the inner channel, a deployed configuration, where the terminal end of the at least one cutting element is located distal to the leading end in the distal direction, and a cutting configuration where the terminal end of the at least one cutting element is angularly offset with respect to the central axis.

5. (canceled)

6. The excision catheter system of claim 2, wherein the at least one cutting element includes an elongate shaft and a cutting hook that extends from the elongate shaft, wherein the cutting hook has a leg that is angularly offset with respect to the elongate shaft.

7. (canceled)

8. The excision catheter system of claim 2, wherein the leading end of the cutting catheter includes a piercing tip configured to pierce a leaflet of a valve.

9. The excision catheter system of claim 4, wherein the at least one cutting element is a pair of cutting elements.

10. The excision catheter system of claim 6, wherein the cutting hook has a curved body that curves around a central axis of the cutting catheter, the curved body defining a forward cutting edge and a rearward cutting edge.

11. (canceled)

12. The excision catheter system of claim 9, wherein the pair of cutting elements are aligned when in the insertion configuration and distal-most terminal ends are splayed apart when in the cutting configuration.

13. (canceled)

14. The excision catheter system of claim 4, further comprising one or more actuators, the one or more actuators configured to cause the at least one cutting element to transition from the insertion configuration into the deployed configuration, and separately to cause the at least one cutting element to transition between the deployed configuration and the cutting configuration.

15.-22. (canceled)

23. The excision catheter system of claim 2, further comprising a retention member configured to retain an excised leaflet.

24.-35. (canceled)

36. The excision catheter system of claim 23, wherein the extraction catheter is a steerable catheter.

37.-48. (canceled)

49. The excision catheter system of claim 2, further comprising an actuator configured to cause the extraction member to transition between the retracted configuration and the expanded configuration.

50. The excision catheter system of claim 2 wherein the at least one cutting element is an electrode that is responsive to electrical energy.

51. The excision catheter system of claim 3, wherein a leading end of the cutting catheter includes a piercing tip configured to pierce a leaflet of a valve.

52. The excision catheter system of claim 3, wherein the at least one cutting element includes an elongate shaft and a cutting hook that extends from the elongate shaft, wherein the cutting hook has a leg that is angularly offset with respect to the elongate shaft.

53. The excision catheter system of claim 3, wherein the at least one cutting element is a pair of cutting elements.

54. The excision catheter system of claim 53, wherein the pair of cutting elements are aligned when in an insertion configuration and distal-most terminal ends are splayed apart when in a cutting configuration.

55. The excision catheter system of claim 3, further comprising a retention member configured to retain an excised leaflet.

56. The excision catheter system of claim 4, further comprising a retention member configured to retain an excised leaflet.

57. The excision catheter system of claim 56, further comprising an introducer configured to slide within the channel of the extraction catheter, the introducer having a lumen.

58. The excision catheter system of claim 4, wherein the leading end of the cutting catheter includes a piercing tip configured to pierce a leaflet of a valve.

59. The excision catheter system of claim 4, wherein the at least one cutting element includes an elongate shaft and a cutting hook that extends from the elongate shaft, wherein the cutting hook has a leg that is angularly offset with respect to the elongate shaft.

60. The excision catheter system of claim 4, wherein the at least one cutting element is a pair of cutting elements.

61. The excision catheter system of claim 60, wherein the pair of cutting elements are aligned when in the insertion configuration and distal-most terminal ends are splayed apart when in the cutting configuration.
